(19) United States
(12) Patent Application Publication
(10) Pub. No.: US 2008/0013047 A1
(43) Pub. Date: Jan. 17, 2008

(54) Diagnostic and Therapeutic System for Eccentric Viewing

(75) Inventors: David P. Todd, Boynton Beach, FL (US); Sigrid Kenkel, Boca Raton, FL (US); Patricia Schulz, Boca Raton, FL (US); Dorothee Schlueter, Sulzetal (DE)

Correspondence Address: BROMBERG & SUNSTEIN LLP, 125 SUMMER STREET, BOSTON, MA, 02110-1618, US

(73) Assignee: NOVAVISION, INC., Boca Raton, FL (US)

(21) Appl. No.: 11/771,293

(22) Filed: Jun. 29, 2007

Related U.S. Application Data
(60) Provisional application No. 60/817,898, filed on Jun. 30, 2006.

(51) Int. Cl. A61B 3/024; A61H 5/00

(52) U.S. Cl. 351/203; 351/224

(57) ABSTRACT

A method is provided to map the sight perception of a patient and/or for therapeutically stimulating the patient. The method includes providing a target focal stimulation on a luminous background stimulation field so that the patient may focus upon the target and thereby keep the patient's retina in a fixed position. A temporary peripheral visual stimulation is then created in a region that is peripheral to the fixation target. The peripheral visual stimulation is darker than the background field. A computer records whether the patient was able to see the peripheral stimulation and stores the patient response data in a manner that maintains association with the position of the peripheral visual stimulation. The process is then repeated with additional stimuli and response recordations to create automatically, in computer media, a peripheral vision map. The vision map may be used to allocate a finite number of therapeutic stimuli to an identified visual field region of the patient having high therapeutic potential. The vision map may include a contrast dimension.

### PRIORITY CLAIM

This application claims priority to U.S. Provisional Application Ser. No. 60/817,898, entitled “Diagnostic and Training System for Eccentric Viewing,” filed Jun. 30, 2006, and which is hereby incorporated in its entirety by reference herein.

### TECHNICAL FIELD

The present invention relates to a system for evaluating and improving eyesight.

### BACKGROUND

The macula is the region of the retina that is used for high acuity vision, as is typically required for reading. Patients suffering from macular damage (e.g., Age-related Macular Degeneration or AMD) may undergo eccentric viewing therapy to regain the ability to recognize objects and text by using peripheral regions of the retina.

To diagnose macular damage, a patient may undergo various types of examinations, including automated perimetry or campimetry, in which the patient is positioned in front of a test surface and is asked to maintain focus on a fixation target. A computer actuates a light source or other visual stimulus to present at a specific point on the test surface. The patient is asked to actuate a data entry device in response to perceived test stimuli and the examiner or computer records the patient input and associated spatial information. In this way, a visual field map is created.

### SUMMARY OF THE INVENTION

In a first embodiment of the invention, a computer is used to map the sight perception of a patient and/or for therapeutically stimulating the patient. The method includes providing a target focal stimulation on a luminous background stimulation field so that the patient may focus upon the target and thereby keep the patient's retina in a fixed position. A temporary peripheral visual stimulation is then created in a region that is peripheral to the fixation target. The peripheral visual stimulation is darker than the background field. A computer records whether the patient was able to see the peripheral stimulation and stores the patient response data in a manner that maintains association with the position of the peripheral visual stimulation. The process is then repeated with additional stimuli and response recordations to create automatically, in computer media, a peripheral vision map.

The background, focal, and peripheral stimuli may be provided by a computerized display. By varying the contrast between the peripheral stimuli and the background stimulation-field during the mapping process, a multidimensional perception map may be created that has at least two spatial dimensions and a contrast dimension.

A target retinal region may be selected for corrective treatment based on the output of the mapping procedure. For example, the target retinal region may be an area that is selected by comparing contrast values in the visual perception map to a threshold contrast value.

Improved precision and accuracy may be obtained by fixing the distance and angle of the patient's head with respect to the stimuli. For example, the patient's eyes may be fixed with a head-positioning device, such as a chinrest attached to a computer display. The user may be provided with a data entry device for activation in response to visual perception of the peripheral test stimuli. In order to maintain and record a patient's continued gaze at the target focal stimulus, the computer may change the focal stimulus (fixation target) and a user may be asked to record those changes.

The data output of a perimetry testing procedure of the first embodiment may be to determine the peripheral retinal region(s) that receives the treatment. The perimetry method used may be the mapping procedure of the above first embodiment. The temporary peripheral visual stimuli may be allocated to positions peripheral to the focal stimulation that are selected based upon data output of a testing procedure so as to bias the allocation of stimuli to target visual field regions determined to have a high potential responsiveness to therapeutic stimulation. To increase precision and accuracy in selecting the peripheral region for treatment, the mapping and treatment procedures of the above embodiments may be performed while fixing the head of a patient in substantially the same fixed position with respect to the stimuli.

In another embodiment of the invention, a computer is used to map the visual sensitivity of the central field of a patient. The method includes providing a visible landmark target on a background field so that the patient may focus upon the target and thereby keep the patient's retina in a determined position. Various transient peripheral visual stimulations are then presented within a region that is peripheral to the fixation target. The peripheral visual stimuli increase incrementally in their contrast to the background medium in order to identify the viewer's visual sensitivity at that test point. The stimulus may be presented as darker than the background field (e.g. a light grey stimulus on a white background incrementally growing blacker) or may be lighter than the background field (e.g. a dark grey stimulus on a black background incrementally growing whiter). A computer records whether the patient was able to see the peripheral stimulation and at what degree of contrast, and then stores the patient response data in a manner that maintains association with the position and contrast of the peripheral visual stimulation. The process is then repeated with additional stimuli and response recordations to create automatically, in computer media, a peripheral vision map.

In another embodiment of the invention, a computer is used to treat a patient for improved visual perception, such as may be obtained with eccentric viewing. The method includes providing a target focal stimulation on a background stimulation field so that the patient may focus upon the target and thereby keep the patient's retina in a determined position. A transient peripheral visual stimulation is then presented in a region that is peripheral to the fixation target. The peripheral visual stimulation differs in contrast from the background field by being lighter or darker. A computer records if, and at what level of contrast, the patient was able to see the peripheral stimulation and stores the patient response data in a manner that maintains an association with the position of the peripheral visual stimulation. The process is then repeated with additional stimuli and response recordations to a visual perception map data set. The computer system may use the map data set to select a region for therapeutic stimuli and actuate a series of therapeutic stimulations biased to that region.

In an embodiment of the invention, a device is used for therapeutic stimulation of the visual field of a patient. The device includes a source of focal stimulation, a source of negative relative luminance peripheral stimuli and a computer system. The computer system includes a processor and computer-executable instructions. The system is adapted to accept a visual field map data set, use the data set to select a peripheral region for therapy, and to repeatedly create peripheral visual stimulation in the selected region. The device may allocate a finite number of stimuli so as to create a bias toward the selected region and may allocate the majority of the stimuli to the region. The system may be adapted to determine and record, to computer media, the presence or absence of the patient's visual perception in response to the peripheral visual stimulations, so as to update the visual field map data set. The updated visual field map may be used to update the region selected for therapy.

In yet another embodiment of the invention, a computer related-medium has computer-executable instructions for performing a method that includes providing, a target focal stimulation on a luminous background stimulation field for the patient to visually fixate upon, creating a temporary peripheral visual stimulation that is darker than the background field in a region peripheral to the focal stimulation, determining and recording to computer media the presence or absence of the patient's visual perception in response to the peripheral visual stimulation, and repeating the steps of creating the stimulation and determining visual perception while varying the position of the peripheral visual stimuli to create automatically, in computer media, a peripheral vision map.

In a related embodiment, the computer-related medium includes instructions for performing a method that includes using the peripheral vision map to allocate a finite number of therapeutic stimuli to an identified visual field region of the patient. The identified visual field region may be a region of high therapeutic potential. The map may be a multi-dimensional map that includes a contrast dimension.

### BRIEF DESCRIPTION OF THE DRAWINGS

The foregoing features of the invention will be more readily understood by reference to the following detailed description, taken with reference to the accompanying drawings, in which:

FIG. 1 is a flow diagram showing a visual testing method in accordance with an embodiment of the invention;

FIG. 2 is a flow diagram showing a visual therapy method in accordance with another embodiment of the invention;

FIGS. 3-5 show screen-shots of a computer program in accordance with an embodiment of the present invention.

### DETAILED DESCRIPTION OF SPECIFIC EMBODIMENTS

Definitions. As used in this description and the accompanying claims, the following terms shall have the meanings indicated, unless the context otherwise requires:

“Brightness” means a level of illumination measured with reference to an absolute quantity associated with an illuminated surface or to its visual perception; and “Luminance” means a level of brightness weighted by the spectral response of the human eye, as described by Born and Wolf, Principles of Optics, 7^{th} edition, Cambridge University Press, 1999, pp. 194-198.

In illustrative embodiments of the present invention, a testing procedure has a patient or other user visually fixate on a visual fixation target stimulus (hereinafter a “target”) while a computer displays peripheral visual stimuli and records user input related to visual perception of the stimuli. In this way, the computer builds a visual field map. An eccentric viewing treatment procedure may then be performed by stimulating areas of the retina identified by the testing procedure. These procedures and devices for implementation of the procedures may be used to treat patients with visual problems such as macular degeneration. The techniques may also prove to have benefit to patients with other visual disorders such as: optic nerve damage, glaucoma, and other retinal problems. Additionally, the techniques may improve peripheral acuity in optically healthy humans; the techniques may be used, for example, by air traffic controllers, military personnel and airport baggage screeners.

The testing and treatment procedures may feature a high degree of flexibility in the types of test stimuli presented; for example, colors, shapes, and contrast ratios may be changed, combinations of shapes and characters and words or sentences may be used as stimuli. The stimuli may be static, moving, scrolling, or have other dynamic effects. The testing and therapy programs may be tailored for a specific patient's known or estimated level of global or localized visual function.

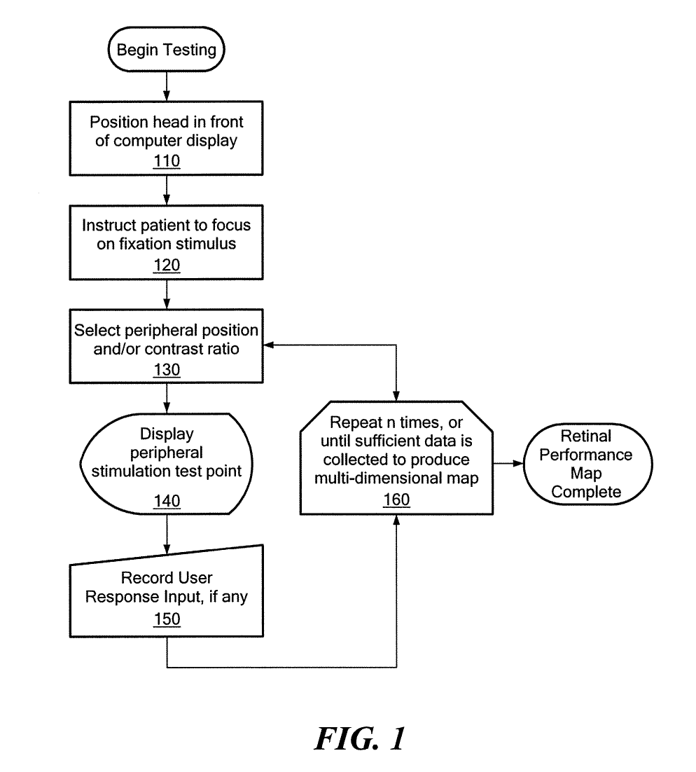

FIG. 1 shows a flow diagram for a perimetry or campimetry testing method in accordance with an embodiment of the invention. A patient's head is positioned (step 110) in front of a computerized display, for example a standard CRT or LCD display. To increase accuracy and reproducibility, the distance and angle of orientation of the patient's head with respect to the display may be mechanically constrained. One example of such a head-positioning device includes a head and chinrest and is disclosed in co-pending U.S. patent application Ser. No. 11/640,548, attorney docket No. 2890/116, filed Dec. 18, 2006, entitled “Adjustable device for vision testing and therapy”, and hereby incorporated by reference in its entirety herein. Another head-positioning device is disclosed in co-pending U.S. patent application Ser. No. 11/153,250, attorney docket No. 2890/105, filed Jun. 15, 2006, entitled: Method and Device for Guiding the Head of a User During Vision Training”, hereby incorporated by reference in its entirety herein. Using this or another head positioning device should help to produce accurate, reproducible maps, which can be used in eccentric vision therapy procedures when the head is similarly positioned. Alternately, accuracy and reproducibility should be achievable with a head-mounted display, such as disclosed in U.S. patent application Ser. No. 11/394,154, attorney docket number 2890/111, filed Mar. 30, 2006 and entitled “Method and Device for Delivering Visual Stimuli with Head Mounted Display During Vision Training”, hereby incorporated by reference in its entirety herein.’

The patient is instructed by the computer program or health care provider to fix their gaze upon a target fixation stimulus (step 120). The fixation target can be, for example, a square, ring or circle permanently positioned on the computer display. The computer display may have a high level of brightness or luminance, and the fixation target may be darker than the background. Alternately, the background may be substantially black with a substantially more luminous fixation target, or may utilize contrasting colors. The computer then selects a region peripheral to the target in which to present a peripheral visual stimulus (step 130). The stimulus may be, for example, a darkened dot on a white display background, a luminous object on a dark background, or a colored object on a background of a contrasting color. The region may be selected from a list, selected randomly, or selected randomly and filtered (e.g., displayed only if the randomly selected region fits with predetermined constraints). If the computer display has a highly luminous background, stimulus of varying darkness (and thus varying contrast ratio) may be used; in this case, the computer will also select the darkness level for the given stimulus.

The peripheral visual stimulus is then presented on the display (step 140). By flashing the stimulus only briefly (e.g., for a duration of 200 ms,) the patient will have insufficient time to avert their gaze, so the retina will remain centered around the target, helping to ensure accurate testing results. Alternately, the stimulus may be flashed for a longer time and the patient induced to re-fixate on the target.

The patient is instructed to respond to perception of the flashed peripheral stimuli (step 150). For example, if the patient sees the stimulus, the patient may press a keyboard button, touch screen, mouse button, give a voice command for automatic speech recognition, make a gesture or use any other suitable computer input method. Touch-screens have the advantage of being able to rapidly record positional information and thus may be used to confirm the accuracy of a patient response. The touch-screen may be of the type that may be operated only with a stylus, finger, or both. Patients with physical handicaps may require special input devices, for example, mouth-actuated or foot-actuated devices.

The process of FIG. 1 is repeated for a determined number of iterations, or until sufficient data is collected to constitute a visual field map of sufficient detail (step 160). In an embodiment, the computer display is segmented into a grid of “cells” and stimuli are successively presented to each of the cells in random or pseudo-random order and the patient's response is recorded. This process may then be repeated. In a specific example, the patient is given a 30 minute break after all cells have been tested and all the cells are tested three times. The data from multiple testing of cells may be aggregated, e.g., by determining the mean or modal response of each cell.

If peripheral stimuli of varying contrasts are used, the map produced will be three dimensional, having two spatial coordinates, and a contrast (sensitivity) coordinate. Such a three dimensional map may be referred to as a “photopic contrast sensitivity function,” or CSF. CSF testing with a light background is especially useful, since it is a much more sensitive test of visual edge detection. As a result, the testing method of FIG. 1 should better reveal certain subtle or early eye disease states than traditional white stimulus presented on a dark background.

One way to build a CSF data set is to gradually increase the contrast presented to the patient (e.g., darken the displayed peripheral stimulus) until detected by the patient. The contrast may be increased while a stimulus is displayed, or increased globally so that the absolute or mean contrast is increased while stimuli flash at various peripheral points. Such a systematic approach is, however, not necessary to arrive at a three dimensional map—a random sampling should also work. Alternate or additional dimensions may also be included, for example, patient response time.

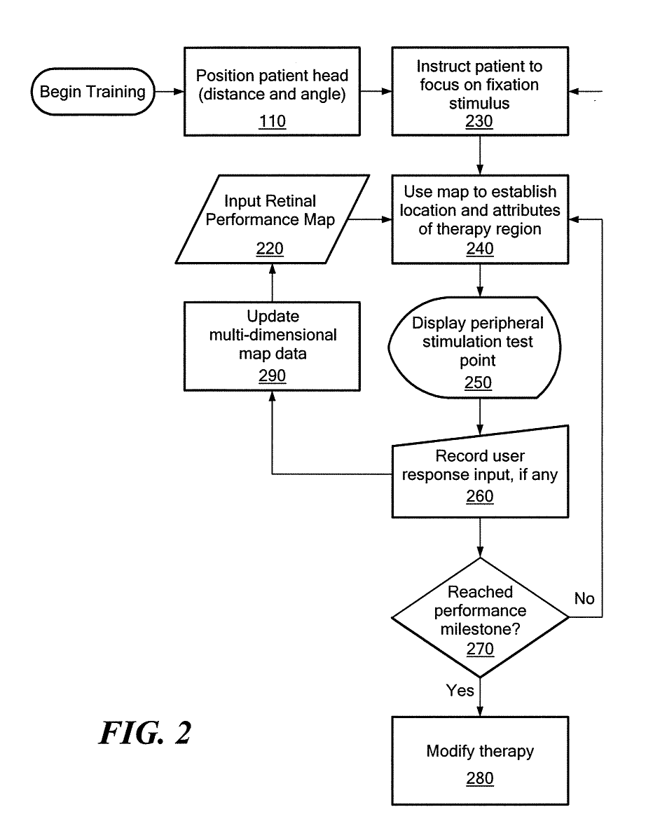

FIG. 2 shows a flow diagram for an automated eccentric-viewing therapy procedure in accordance with an embodiment of the present invention. The therapy procedure utilizes a computer (including storage media, processor, and display driver), a computerized display and computer input device. To begin, the patient's head is positioned (step 110) at a fixed distance and angle. By using the same head position that was used for the testing procedure (step 110 of FIG. 1), the testing information may be used with maximal utility in a subsequent therapy procedure. Accordingly, the therapy procedure may be performed with a head positioning system and display that is the same, or substantially identical to, the system used for the testing procedure of FIG. 1.

After the patient's head is positioned, the patient is instructed to focus on a fixation target (step 230). The fixation target location and features may need to be adjusted to be appropriate for the patient's central vision function; for example, a patient with exceptionally poor vision may require a larger target. A retinal performance map, such as the output of the procedure of FIG. 1, is used as initial input (step 220) to an algorithm for selecting a position (and optionally, a contrast ratio) on the computer display to display a peripheral visual stimulation (step 240). The display positions at which stimuli are presented are based on a determination of which visual field regions will be most responsive to the stimulative therapy. For example, a set of rules may be used to assign the various display cells to an intact zones, transition zones, zones of deteriorated vision, zones of residual vision, and blind zones and one or more of these may be targeted, fully or partially. Zones, loci or other defined regions may be targeted by biasing the number of stimuli out of the total number of stimuli presented that are allocated to that display cells associated with that zone. For example, all of the stimuli may be targeted to a zone, or the majority of the stimuli may be targeted to a zone. In a specific embodiment, a fraction of the stimuli are also allocated to additional cells that have not been identified as potentially responsive. The additional cells may be selected, without limitation, from the set of all the cells, from cells neighboring the targeted zones, or from all the cells not targeted. Stimulating the additional cells allows for the collection of additional test data, which can be used to verify that the visual field map continues to be accurate and to track changes in the patient's responsiveness over time. Any changes detected may be used to redefine the stimulus allocation; for example, by recruiting (i.e., increasing stimulative allocation) new cells that are determined to have a high therapeutic potential or dropping (i.e., reducing stimulative allocation) to cells that are determined to have low therapeutic potential. In this way, the therapy may be repeatedly or continuously adapted to maximize effectiveness.

As in the testing procedure, the stimulation may be a dark spot on a luminous (e.g., white) background that is flashed for less than 1 second. After the stimulation is presented (step 250), the user records visual perception of the stimulation, if any (step 260). As for the testing procedure of FIG. 1, patient fixation may be verified by monitoring their response to subtle and rapid changes in the fixation stimulus that are computer-actuated at random or pseudo-random times throughout the therapy procedure; this helps ensure proper retinal targeting of the therapeutic stimuli. Based on their response, feedback in the form of reward or praise may be provided to the patient to encourage proper fixation.

Optionally, the visual perception data may be used to update the map (step 290), either continuously or intermittently. By recording and analyzing the user responses, the computer can determine if the patient has reached a particular level of visual performance (step 270). Optionally, if a benchmark or milestone level of performance has been reached, the computer can provide a different, or more advanced, level of therapy (step 280). If the milestone has not been achieved, or to cement gains, the therapy is continued from step 240 by selecting another display position for another peripheral stimulus. Alternately, if performance is not increasing, the level may be reduced. Of course, a patient may be able to terminate the program at any time, and the computer will maintain a record of performance for analysis by a healthcare professional or for setting a difficulty level for future therapy sessions. The user may also be able to pause the program to take a break.

Alteration of the difficultly level may include changing the peripheral stimulus. By using dark stimuli on a light background (white or lightly colored), a wide variety of stimuli may be presented. Examples of various types of peripheral stimuli that may be displayed by a computer on a light background include:
black spots (e.g., circles, squares or other small shapes)
grey squares of varying contrast ratios
other simple patterns
letters and/or characters
words, or groups thereof
scrolling words, scrolling groups of words

In a related embodiment, therapeutic stimuli are targeted to a particular retinal sub-region. This sub-region may be adaptively modified based on campimetric data recorded as part of intermittent test sessions during therapy. For example, the size of the region may be decreased based on recorded shrinkage of a zone of deteriorated vision as recorded via the user input device.

The use of a multidimensional map may allow selection of a region of the retina for treatment that may be different than the regions of high-sensitivity that would be typically identified by conventional perimetry. For example, selection of the region for treatment could be based on parameters such as the degree of response to contrast, or patient response times for different retinal regions. Alternately or in addition, selection of the region for treatment could be based on position relative to the impaired area of the visual field.

In either the testing or therapy procedures, the computer system may record and analyze statistical information regarding patient performance and may associate this information with other information about the patient (e.g., demographic or health information). Such statistical information may include false negative and false positive responses, and mean or median response times and may be global or segmented based on retinal region. The system may provide additional levels of analysis such as graphs, charts, and trend information. The various statistical information may be used to adjust treatment (e.g., level or visual region) or to provide or adjust incentives to patients (e.g., reward points, scores or praise).

In embodiments, the devices, methods and algorithmic embodiments of the methods may utilize dynamic target fixation stimuli and/or dynamic peripheral stimuli. Such dynamic stimuli are the subject of co-pending U.S. Patent Application No. 60/867,449 for “: Dynamic Fixation and Peripheral Stimuli for Visual Field Testing and Therapy”, attorney docket No. 2890/114, filed Nov. 28, 2006 and hereby incorporated in its entirety herein. Additional disclosure regarding therapeutic stimulation devices and methods are given in U.S. Pat. No. 6,464,356 and U.S. Patent Application Publication No. 2005-0213033, both to Sabel; both are incorporated herein by reference in their entirety.

### EXAMPLE 1

A therapeutic procedure uses the following steps:
The patient is given a fixation target stimulus and asked to respond to changes in the fixation stimulus by actuating an input device. In this way, the patient is encouraged to maintain fixation upon the target fixation stimulus and deviations from fixation may be recorded.
Level 1: The computer program delivers stimuli to a targeted locus within the visual field for eccentric vision therapy.
Once the patient has achieved a pre-determined level of accuracy in the stimulus detection task, the patient is ready for Level 2.
Level 2—character recognition: The program delivers an auditory cue referring to the correct stimulus to follow (this feature is optional). A series of characters are then presented. The patient is asked to identify the character in the series that matches the auditory cue and, in response, activate an input device (e.g., press a mouse-button). For example, the auditory cue may announce, “identify the animal”; this is followed by stimuli in the form of the words fat, cap, cat, and far.
This process may be completed over multiple levels and the difficulty of the task is increased based on performance of the patient.

### EXAMPLE 2

### A Testing Procedure

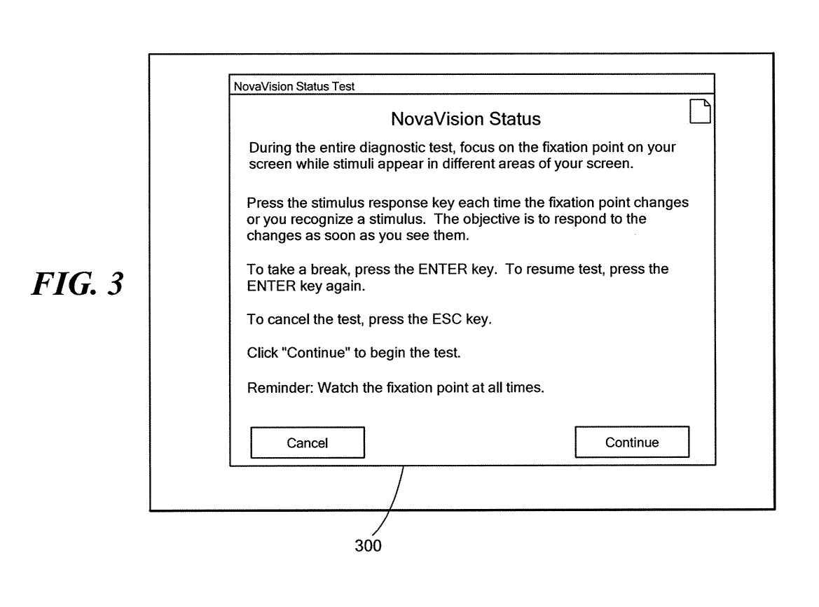

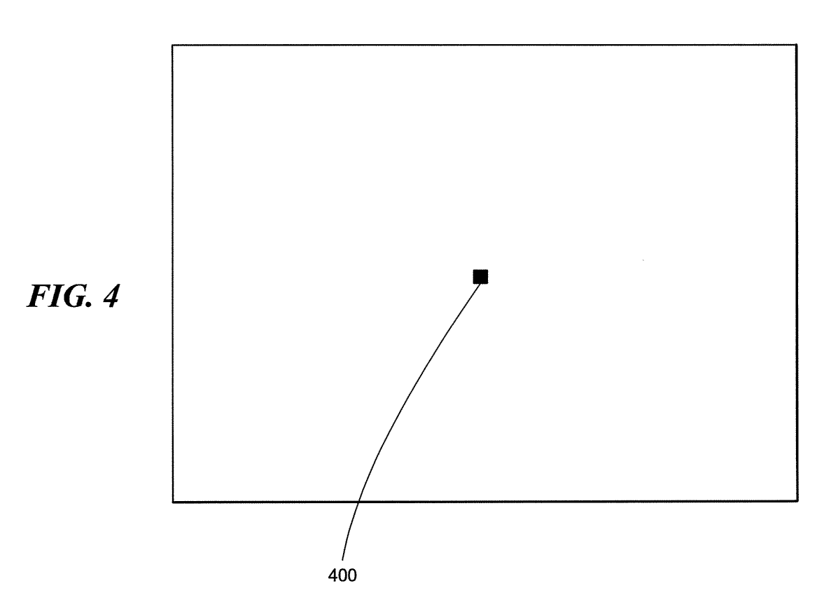

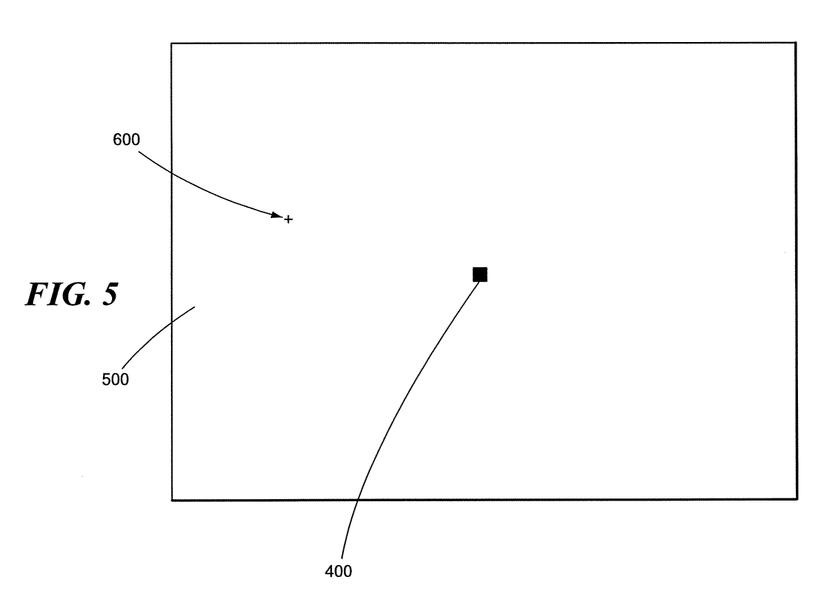

FIGS. 3-5 show screen-shots of a computer-delivered diagnostic test demonstration in accordance with an embodiment of the invention. Instructions 300 are delivered to a user on a computer monitor; the user is instructed to focus on the fixation point (target) 400, which is displayed on a luminous white background 500, and to press a key each time the fixation point changes or a peripheral stimulus 600 is flashed. The test may also be paused or canceled with a keystroke. The background remains white for the majority of the testing time with intermittent flashes of stimuli 600 in varying peripheral regions.

In alternative embodiments, the disclosed methods for visual testing and therapy may be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems.

Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

The described embodiments of the invention are intended to be merely exemplary and numerous variations and modifications will be apparent to those skilled in the art. All such variations and modifications are intended to be within the scope of the present invention as defined in the appended claims.

What is claimed is:

1. A method for mapping sight perception, or for therapeutic stimulation, of a patient, the method comprising:
(a) providing, for the patient to visually fixate upon, a target focal stimulation on a luminous background stimulation field;
(b) creating a temporary peripheral visual stimulation in a region peripheral to the focal stimulation, the peripheral visual stimulation being darker than the background field;
(c) determining, and recording to computer media, the presence or absence of the patient's visual perception in response to the peripheral visual stimulation; and
(d) repeating steps (b) and (c) while varying the position of the peripheral visual stimuli to create automatically, in computer media, a peripheral vision map.

2. A method according to claim 1, wherein step (d) further comprises varying the contrast between the peripheral stimuli and the background stimulation field to create a data set of at least two spatial dimensions and one contrast dimension.

3. A method according to claim 1, further comprising selecting a retinal region to receive a corrective treatment.

4. A method according to claim 2, further comprising selecting a retinal region for corrective treatment.

5. A method according to claim 4, wherein the retinal region is selected based on comparison of contrast values in the data set to a threshold contrast value.

6. A method according to claim 1, further including using a head-positioning device to fix the position of a patient's head in relation to the stimuli.

7. A method according to claim 1, wherein one of the background, target and peripheral stimuli are created using a computerized display.

8. A method according to claim 1, wherein determining the presence of absence of the patient's visual perception further comprises providing a user-activated computer data entry device for the patient to activate in response to visual perception.

9. A method according to claim 1, further comprising changing the target stimulus at a predetermined time.

10. A method according to claim 9, further comprising instructing the user to record changes in the target stimulus for purposes of maintaining and recording gaze fixation.

11. A method according to claim 10 further comprising recording the user's response to changes in the target stimulus.

12. A method for improving the eyesight of a patient, the method comprising:
(a) providing, for the patient to visually fixate upon, a target focal stimulation on a luminous background stimulation field;
(b) creating a temporary peripheral visual stimulation in a region peripheral to the focal stimulation, the peripheral visual stimulation being darker than the background field;
(c) determining, and recording to computer media, the presence or absence of the patient's visual perception in response to the peripheral visual stimulation; and
(d) repeating steps (b) and (c) while varying the position of the peripheral visual stimuli.

13. A method according to claim 12, wherein the temporary peripheral visual stimuli are allocated to positions peripheral to the focal stimulation that are selected based upon data output of a testing procedure so as to bias the allocation of stimuli to target visual field regions determined to have a high potential responsiveness to therapeutic stimulation.

14. A method according to claim 13, wherein the testing procedure comprises:
(a) providing, for the patient to visually fixate upon, a target focal stimulation on a luminous background stimulation field;
(b) creating a transient peripheral visual stimulation in a region peripheral to the focal stimulation, the peripheral visual stimulation being darker than the background field;
(c) determining, and recording to computer media, the presence or absence of the patient's visual perception in response to the peripheral visual stimulation; and
(d) repeating steps (b) and (c) while varying the position of the peripheral visual stimuli to create, in computer media, a peripheral vision map.

15. A method according to claim 14, wherein the testing procedure and eyesight improving method are performed with the patient's head constrained in substantially the same fixed position relative to the stimuli.

16. A device for mapping peripheral vision sensitivity and function or for delivering therapeutic stimuli, the device comprising:
a source of focal stimulation;
a source of negative relative luminance peripheral stimuli;
a computer system including a processor and computer-executable instructions the system adapted to repeatedly create the peripheral visual stimulation in a region peripheral to the focal stimulation, and to determine and record, to computer media, the presence or absence of the patient's visual perception in response to the peripheral visual stimulations, so as to create, in the computer media, a visual perception map data set.

17. A device according to claim 16 wherein the computer system is operable varying the contrast between the peripheral stimuli and the background stimulation field to create a visual perception map of at least two spatial dimensions and one contrast dimension.

18. A device according to claim 16, wherein the computer system is operable to select a region adjacent to a region of impaired vision to receive the peripheral stimuli.

19. A device according to claim 18, wherein the region is selected based on comparison of contrast values in the visual perception map to a threshold contrast value.

20. A device according to claim 19, further including a head positing means to fix the position of a patients head to the peripheral stimuli.

21. A device according to claim 16, wherein the background, focal and peripheral stimuli are created using a computerized display.

22. A device according to claim 16, further comprising a user activated computer data entry device for the patient to activate in response to visual perception so as to record the presence or absence of the patient's visual perception in response to the peripheral stimuli.

23. A device according to claim 16, wherein the computer system uses the map data set to select a region for therapeutic stimuli and actuates a series of therapeutic stimulation biased to that region.

24. A device for therapeutic stimulation of the visual field of a patient, the device comprising:
a source of focal stimulation;
a source of negative relative luminance peripheral stimuli;
a computer system including a processor and computer-executable instructions the system adapted to accept visual field map data set, use the map data to select a peripheral region for therapy, and to repeatedly create peripheral visual stimulation in the region.

25. A device according to claim 24, wherein creating the peripheral stimulation in the region includes allocating a finite number of stimuli so as to create a bias toward the region.

26. A device according to claim 25, wherein creating a bias toward the region includes allocating a majority of the stimuli to the region.

27. A device according to claim 24, wherein the computer is adapted to determine and record, to computer media, the presence or absence of the patient's visual perception in response to the peripheral visual stimulations, so as to update the visual field map data set.

28. A device according to claim 26, wherein the updated visual field map is used to update the region selected for therapy.

29. A computer related-medium having computer-executable instructions for performing a method comprising:
(a) providing, for the patient to visually fixate upon, a target focal stimulation on a luminous background stimulation field;
(b) creating a temporary peripheral visual stimulation in a region peripheral to the focal stimulation, the peripheral visual stimulation being darker than the background field;
(c) determining, and recording to computer media, the presence or absence of the patient's visual perception in response to the peripheral visual stimulation; and
(d) repeating steps (b) and (c) while varying the position of the peripheral visual stimuli to create automatically, in computer media, a peripheral vision map.

30. A computer-related medium according to claim 29 further comprising instructions for performing a method that further comprises:
(e) using the peripheral vision map to allocate a finite number of therapeutic stimuli to an identified visual field region of the patient.

31. A computer-related medium according to claim 30, wherein the identified visual field region is a region of high therapeutic potential.

32. A computer-related medium according to claim 29 wherein the map is a multidimensional map that includes a contrast dimension.
